(19) United States
(12) Patent Application Publication
(10) Pub. No.: US 2023/0138414 A1
(43) Pub. Date: May 4, 2023

(54) METHODS AND COMPOSITIONS FOR PREVENTING AND TREATING MYOPIA

(71) Applicant: The Trustees of Columbia University in the City of New York, New York, NY (US)

(72) Inventors: Andrei V. Tkatchenko, Rye, NY (US); Tatiana V. Tkatchenko, Rye, NY (US)

(21) Appl. No.: 18/073,181

(22) Filed: Dec. 1, 2022

Related U.S. Application Data
(63) Continuation-in-part of application No. PCT/US2021/035344, filed on Jun. 2, 2021.
(63) Continuation-in-part of application No. PCT/US2021/036614, filed on Jun. 9, 2021.
(63) Continuation-in-part of application No. PCT/US2021/036617, filed on Jun. 9, 2021.
(63) Continuation-in-part of application No. PCT/US2021/036628, filed on Jun. 9, 2021.
(63) Continuation-in-part of application No. PCT/US2021/036738, filed on Jun. 10, 2021.
(63) Continuation-in-part of application No. PCT/US2021/036741, filed on Jun. 10, 2021.
(60) Provisional application No. 63/033,291, filed on Jun. 2, 2020.
(60) Provisional application No. 63/037,891, filed on Jun. 11, 2020.
(60) Provisional application No. 63/037,901, filed on Jun. 11, 2020.
(60) Provisional application No. 63/037,910, filed on Jun. 11, 2020.
(60) Provisional application No. 63/037,918, filed on Jun. 11, 2020.
(60) Provisional application No. 63/037,920, filed on Jun. 11, 2020.

(51) Int. Cl. A61K 31/451; A61K 31/137; A61K 31/165; A61K 31/4375; A61K 31/198; A61P 27/10

(52) U.S. Cl. CPC A61K 31/451; A61K 31/137; A61K 31/165; A61K 31/198; A61K 31/4375; A61P 27/10

(57) ABSTRACT

The disclosure relates to methods and compositions for preventing and/or treating an ocular disease. In particular, the disclosure relates to preventing and/or treating myopia with systemic or topical administration of an active drug compound including levocabastine hydrochloride, which is a selective histamine H1-receptor antagonist; fenoterol hydrobromide, which is a β₂-adrenergic receptor agonist; trichostatin A, which is a histone deacetylase (HDAC) inhibitor; fingolimod (FTY720) phosphate, which is a sphingosine-1-phosphate receptor modulator; berberine chloride, which is a berberidaceaen alkaloid; S-methyl-L-thiocitrulline hydrochloride, which is a selective neuronal nitric oxide synthase (nNOS) inhibitor; and derivatives thereof.

### CROSS REFERENCE TO RELATED APPLICATION

The present application is a continuation-in-part of PCT Application No. PCT/US2021/035344, filed Jun. 2, 2021, which claims priority to U.S. Pat. Application Serial No. 63/033,291, filed Jun. 2, 2020; and this application is a continuation-in-part of PCT Application No. PCT/US2021/036614, filed Jun. 9, 2021, which claims priority to U.S. Pat. Application Serial No. 63/037,891, filed Jun. 11, 2020; and this application is a continuation-in-part of PCT Application No. PCT/US2021/036617, filed Jun. 9, 2021, which claims priority to U.S. Pat. Application Serial No. 63/037,901, filed Jun. 11, 2020; and this application is a continuation-in-part of PCT Application No. PCT/US2021/036628, filed Jun. 9, 2021, which claims priority to U.S. Pat. Application Serial No. 63/037,910 filed Jun. 11, 2020; and this application is a continuation-in-part of PCT Application No. PCT/US2021/036738, filed Jun. 10, 2021, which claims priority to U.S. Pat. Application Serial No. 63/037,918, filed Jun. 11, 2020; and this application is a continuation-in-part of PCT Application No. PCT/US2021/036741, filed Jun. 10, 2021, which claims priority to U.S. Pat. Application Serial No. 63/037,920 filed Jun. 11, 2020, all of which are hereby incorporated by reference in their entirety.

### FIELD

The disclosure relates to methods and compositions for preventing and/or treating an ocular disease. In particular, the disclosure relates to preventing and/or treating myopia with systemic or topical administration of an active drug compound including: levocabastine hydrochloride, which is a selective histamine H1-receptor antagonist; fenoterol hydrobromide, which is a β₂-adrenergic receptor agonist; trichostatin A, which is a histone deacetylase (HDAC) inhibitor; fingolimod (FTY720) phosphate, which is a sphingosine-1-phosphate receptor modulator; berberine chloride, which is a berberidaceaen alkaloid; S-methyl-L-thiocitrulline hydrochloride, which is a selective neuronal nitric oxide synthase (nNOS) inhibitor; and derivatives thereof.

### BACKGROUND

Myopia (nearsightedness) is the most common ocular disorder in the world. The prevalence of myopia in the U.S. has increased from 25% to 48% in the last 40 years.^{1,2} In parts of Asia, more than 80% of the population are affected by myopia.³ The worldwide prevalence of myopia is predicted to increase from 25% in 2020 to 50% by 2050.⁴ Myopia results in 250-billion-dollar worldwide productivity loss a year.

Myopia often leads to serious pathological complications such as chorioretinal atrophy, retinoschisis, retinal tears, retinal detachment, and myopic macular degeneration, which often lead to blindness.^{5,}⁶ It also represents a major risk factor for a number of other serious ocular diseases such as cataracts and glaucoma, which also often lead to vision impairment and vision loss.^{7,}⁸ Because of the increasing prevalence, myopia is rapidly becoming one of the leading causes of vision loss, and the World Health Organization designated myopia as one of five priority health conditions.^{5,9}

Development of myopia is controlled by both environmental and genetic factors.¹⁰ Human population studies suggest that the leading environmental factors causing human myopia are nearwork and reading,¹¹⁻¹³ which are associated with hyperopic defocus produced by the lag of accommodation, i.e., insufficiently strong accommodative response to near objects when the subject performs nearwork tasks.^{14,}¹⁵ The optical blur produced by the hyperopic defocus is believed to be the signal that drives excessive eye growth and causes myopia.^{16,}¹⁷ For example, analysis of the incidence of myopia in orthodox Jewish students (who spent the majority of the day reading) and secular Jewish students (who spent less time reading) found that the orthodox students had a much higher incidence and degree of myopia as compared to the secular students,¹⁸ which suggests that reading is the factor that causes myopia. In addition, there are a number of epidemiological studies that show that myopia is more common in urban areas, amongst professionals, educated patients, computer users, university students, and associated with increased intelligence.¹⁹⁻²³ Myopia is also increased in individuals who perform tasks requiring increased use of eyes such as microscopists.²⁴ The association between optical defocus and myopia was supported by the numerous animal studies, which found that degradation of visual input using either diffusers or negative lenses causes excessive eye growth and myopia in species as diverse as fish, chickens, tree shrews, monkeys, guinea pigs and mice.²⁵

Although the increase in the prevalence of myopia in recent decades is primarily attributed to rapidly increasing exposure of young children to nearwork,²⁶ the contribution of genetic factors to myopia development has been estimated to be between 60% and 80%.²⁷ The incidence of myopia increases when both parents have myopia.²⁰ Numerous studies have shown that the refractive error of the parents is the most important predictor of the development of myopia.^{28,} ²⁹ Strong support for the role of genetic factors in myopia development also comes from studies comparing monozygotic ³⁰ and dizygotic twins.^{31,} ³² Myopia is a complex genetic disease, which is controlled by hundreds of genes; similar to height and weight.^{27,}³³ Genetic studies have implicated over 900 genes to the development of human myopia.^{27,}³³

Thus, both environmental and genetics factors have been shown to contribute to myopia development.¹⁰ Moreover, a recent study demonstrated the existence of genes, which modulate the impact of myopiagenic environmental factors on refractive eye development.³⁴ Further support for gene-environment interaction in the development of myopia comes from gene-expression-profiling studies which uncovered that development of myopia is accompanied by large-scale changes in gene expression in the eye, suggesting that nearwork activates molecular signaling pathways in the eye which stimulate excessive eye growth leading to the development of myopia.^{33,} ³⁵⁻³⁷ Several studies revealed that the eye responds to local changes in optical defocus with local changes in growth rate, thus suggesting that information about optical defocus is summed up across the entire surface of the retina and the integrated signal regulates the growth of the eye.^{38,}³⁹ Importantly, the eye is able to respond to myopiagenic optical defocus even if the optic nerve was severed,³⁹ demonstrating that the signaling cascade regulating refractive eye development is located within the eye itself and does not require a feedback from the brain.

Myopia seems to progress the most during a susceptible period between ages 6-16 and then begins to slow down.^{40,} ⁴¹ In previous generations, myopic progression was assumed to end at around age 20. However, that has changed since more students have entered graduate school followed by jobs requiring 8 hours of sustained computer work.⁴² This conjecture was recently studied in a cohort of post university graduates with a mean age of 35.⁴³ Myopia was found to progress in approximately 10% of the cohort who spent a lot of time in front of computers. Those subjects who did not spend time in front of computers did not progress as much.

Current approved treatment options for myopia are limited to optical correction using spectacles or contact lenses. Optical correction using single-vision corrective lenses, which is the most widely used treatment option for myopia, does not stop the progression of myopia and does not prevent the blinding pathological complications associated with the disease.^{44,} ⁴⁵ Several experimental optics-based clinical interventions to slow myopia progression, such as spectacles with bifocal lenses, multifocal and Ortho-K contact lenses, have shown some promise; however, these treatment options have low efficacy.⁴⁶

Spectacles with bifocal lenses were the first to be used to control myopia progression. The multi-center COMET study, which was designed to determine if bifocals could slow the progression of myopia as compared to a single vision spectacle lenses demonstrated that bifocals slowed the progression of myopia by 20% in the first year; however, the effect was significantly reduced in years 2-4.⁴⁷

Two separate meta-analyses analyzed the ability of Ortho-K lenses to slow myopic progression,^{48,}⁴⁹ and found that myopic progression can be reduced by approximately 45%; however one study found that there was a considerable rebound effect when Ortho-K lenses were discontinued.⁵⁰

Recently, there has been increasing interest in the use of soft multifocal contact lenses to replicate the optics of Ortho-K.⁵¹⁻⁵³ A meta-analysis, which included 587 subjects from 8 studies found that concentric ring and distance centered multifocal contact lenses slowed myopia progression by 30-38% over 24 months.⁵⁴

Currently available pharmacological options for myopia control are essentially limited to two drugs, atropine and 7-methylxanthine, which have significant side effects and/or relatively low efficacy.

Atropine, a nonselective muscarinic antagonist, is an alkaloid produced by Atropa belladonna, which has been traditionally used in ophthalmic practice as a mydriatic and cycloplegic drug. Several clinical trials have evaluated the effects of different concentrations of atropine on myopia progression and its long-term effects on visual function in children. The first trial, Atropine for the treatment of Myopia 1 (ATOM1), revealed that the 1% atropine eye drops retard the progression of myopia by approximately 76% over the 2-year treatment period.⁵⁵ However, the follow up study found that the discontinuation of treatment led to a strong rebound effect resulting in the 300% increase in the myopia progression rate compared to placebo during the first 12 months after the cessation of atropine, which eliminated approximately 60% of the 2-year treatment effect.⁵⁶ Moreover, 1% atropine caused uncomfortable side effects such as photophobia, reduced accommodation amplitude, and blurred vision. The follow up trial, ATOM2, evaluated the effects of 0.5%, 0.1%, and 0.01% atropine on the progression of myopia in children and found that 0.5% atropine suppressed the progression of myopia by 75%, while 0.1% and 0.01% atropine retarded progression by 68% and 59% respectively.⁵⁷ The cessation of treatment caused a 218% rebound increase in the progression rate compared to placebo in the group treated with 0.5% atropine and 170% increase in the group treated with 0.1% atropine during the first 12 months after stopping the administration of the drug.⁵⁸ However, the progression rate dropped by approximately 30% in the group treated with 0.01% atropine.⁵⁸ These findings were reinforced by the recent 5-year follow up study, which revealed that a higher initial atropine dose predisposed children to greater myopia progression after the cessation of treatment and suggested that 0.01% atropine provides the best long-term outcome with approximately 30% suppression effect.⁵⁹ These findings were refined by a recent trial, Low-Concentration Atropine for Myopia Control (LAMP) study, which suggested that low-dose atropine has a dose-dependent suppressive effect on myopia progression ⁶⁰. This study found that 0.01% atropine retarded progression of myopia by 27% over 1-year period, compared to 43% and 67% achieved with 0.025% and 0.05% atropine respectively. However, a recent study found that the use of atropine in juvenile primates has long-term adverse effects on the development of ocular components and emmetropization, which puts in doubt the utility of atropine as anti-myopia drug.⁶¹

7-methylxanthine (7-MX), a nonselective adenosine receptor antagonist, is a natural metabolite of caffeine and theobromine, two alkaloids produced by several plant species and major constituents of cacao, coffee, and tea. The first indication that 7-MX might be a potential medication for myopia control came from an observation that 7-MX causes thickening of the sclera and an increase in the diameter of the scleral collagen fibrils,⁶² i.e., it causes changes in the sclera opposite to those observed in myopic eyes. A small follow-up clinical trial analyzed the effect of a daily oral consumption of 400 mg (∼15 mg/kg) of 7-MX on the progression of myopia in children and revealed that 7-MX can potentially suppress myopia by approximately 22% in subjects with slow progressing myopia, while had no effect on myopia progression in the subjects with high rates of progression.⁶³ In guinea pigs, a 300 mg/kg dose of 7-MX was shown to suppress myopia by 49%.⁶⁴ Similarly, a 30 mg/kg dose of 7-MX reduced the extent of induced myopia in rabbits by approximately 67%.⁶⁵ Recent data from a study in monkeys also suggested that 7-MX can suppress myopia in primates, but the effect strongly depended on the genetic background of a specific subject.⁶⁶ Thus, preliminary data suggest that 7-MX has therapeutic potential for myopia control in subjects with slow progressing myopia, but the questions of the effective dose and efficacy in humans remain to be clarified. The safety profile and long-term effects of daily oral consumption of 7-MX in children is currently unknown.

Several other compounds have been suggested to suppress myopia to various degrees. The muscarinic receptor antagonists pirenzepine and himbacine were shown to inhibit the development of experimental myopia in tree shrews, rhesus monkeys, and chickens.^{67,}⁶⁸ While pirenzepine was found to suppress the progression of myopia in children by 40%, clinical trials were eventually discontinued due to serious side effects.⁶⁹ Several GABA_{B} and GABAc receptor antagonists, such as (1,2,5,6-tetrahydropyridin-4yl) methylphosphinic acid (TPMPA), CGP46381, and (3-aminocyclopentanyl) butylphosphinic acid (3-ACPBPA) were shown to suppress myopia development in chickens and guinea pigs.⁷⁰⁻⁷² Further, α-adrenergic agonists, such as clonidine and guanfacine, were shown to inhibit experimentally induced myopia in chickens,⁷³ while brimonidine suppressed myopia in chickens⁷³ and guinea pigs.⁷⁴ Moreover, apomorphine, a dopamine receptor agonist, was found to inhibit myopia development in several animal models, such as chicken, mouse and non-human primates,^{75,} ⁷⁶ and an intraocular-pressure-lowering drug latanoprost was found to reduce progression of myopia in guinea pigs.⁷⁷ Finally, a recent drug screen in a mouse model of myopia identified crocetin, a natural carotenoid found in the crocus flowers and Gardenia jasminoides fruits, as a potential anti-myopia agent.⁷⁸

The prevalence of myopia has been increasing exponentially throughout the world in recent years and already reached epidemic proportions in many countries. With the prevalence of myopia projected to increase to 50% of the world’s population by 2050, the world will soon face a public health crisis in vision loss because 8% of low to moderate myopes and 29% of high myopes will develop myopic macular degeneration and will lose sight.⁷⁹ Currently available optics-based treatment options for myopia have low efficacy and can only slow the progression of myopia, but not stop it. Currently available pharmacological options have either low efficacy and/or serious adverse effects. Clearly, there is an urgent medical need to develop a product for myopia control that, compared to the currently available products, can achieve much greater efficacy and can be safely used in children.

### SUMMARY

The disclosure provides a method for preventing and/or treating myopia in a subject in need thereof by suppressing ocular signaling pathways underlying the development of myopia using an oral composition, extended drug release formulations or compositions, extended drug delivery by contact lenses, or eye drops comprising a drug compound or agent identified using pharmacogenomic pipeline for anti-myopia drug development.

Thus, one embodiment is a method of preventing and/or treating myopia in a subject in need thereof comprising administering to the subject a therapeutically effective amount of a composition comprising an active drug compound identified using pharmacogenomic pipeline for anti-myopia drug development.

In one embodiment, the active drug compound is a selective histamine H1-receptor antagonist levocabastine hydrochloride having the structure:

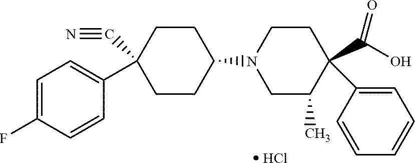

or a derivative thereof.

In some embodiments, the disclosure provides methods for preventing and/or treating myopia by administering to a subject a therapeutically effective amount of levocabastine hydrochloride or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a levocabastine hydrochloride or a derivative thereof, in a form of oral composition, extended drug release formulation or composition, extended drug delivery by contact lenses, or eye drops during a susceptible period for myopia development.

In further embodiments, the active drug compound is a selective histamine H1-receptor antagonist.

In some embodiments, the selective histamine H1-receptor antagonist includes but is not limited to azelastine, alcaftadine, antazoline, carbinoxamine, epinastine, olopatadine, and ketotifen, and derivatives thereof.

Thus, in further embodiments, the disclosure provides methods for preventing and/or treating myopia by administering to a subject a therapeutically effective amount of a selective histamine H1-receptor antagonist, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a selective histamine H1-receptor antagonist, in a form of oral composition, extended drug release formulation or composition, extended drug delivery by contact lenses, or eye drops during a susceptible period for myopia development.

In a further embodiment, the active drug compound is a β2-adrenergic receptor agonist, fenoterol hydrobromide, having the structure:

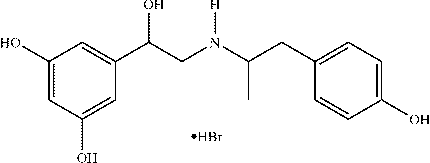

or a derivative thereof.

In some embodiments, the disclosure provides methods for preventing and/or treating myopia by administering to a subject a therapeutically effective amount of fenoterol hydrobromide or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a fenoterol hydrobromide or a derivative thereof, in a form of oral composition, extended drug release formulation or composition, extended drug delivery by contact lenses, or eye drops during a susceptible period for myopia development.

In further embodiments, the active drug compound is a β2-adrenergic receptor agonist.

In some embodiments, the β₂-adrenergic receptor agonist includes but is not limited to, bitolterol, isoprenaline, levosalbutamol, levalbuterol, orciprenaline, metaproterenol, pirbuterol, procaterol, ritodrine, salbutamol, albuterol, terbutaline, arformoterol, bambuterol, clenbuterol, formoterol, salmeterol, and derivatives thereof

Thus, in further embodiments, the disclosure provides methods for preventing and/or treating myopia by administering to a subject a therapeutically effective amount of a β2-adrenergic receptor agonist, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a β2-adrenergic receptor agonist, in a form of oral composition, extended drug release formulation or composition, extended drug delivery by contact lenses, or eye drops during a susceptible period for myopia development.

In a further embodiment, the active drug compound is a histone deacetylase (HDAC) inhibitor, trichostatin A, having the structure:

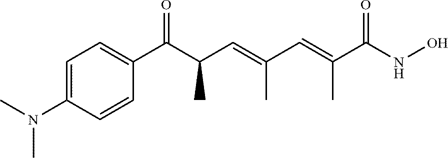 [SMILES: CC(=C\[C@@H](C)C(=O)c1ccc(N(C)C)cc1)/C=C(\C)C(=O)NO]

or a derivative thereof.

In some embodiments, the disclosure provides methods for preventing and/or treating myopia by administering to a subject a therapeutically effective amount of trichostatin A or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a trichostatin A or a derivative thereof, in a form of oral composition, extended drug release formulation or composition, extended drug delivery by contact lenses, or eye drops during a susceptible period for myopia development.

In further embodiments, the active drug compound is an HDAC inhibitor.

In some embodiments, the HDAC inhibitor includes but is not limited to, vorinostat, belinostat, panobinostat, entinostat, and mocetinostat, and derivatives thereof.

Thus, in further embodiments, the disclosure provides methods for preventing and/or treating myopia by administering to a subject a therapeutically effective amount of the HDAC inhibitor, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of the HDAC inhibitor, in a form of oral composition, extended drug release formulation or composition, extended drug delivery by contact lenses, or eye drops during a susceptible period for myopia development.

In a further embodiment, the active drug compound is a sphingosine-1-phosphate receptor modulator, fingolimod (FTY720) phosphate, having the structure:

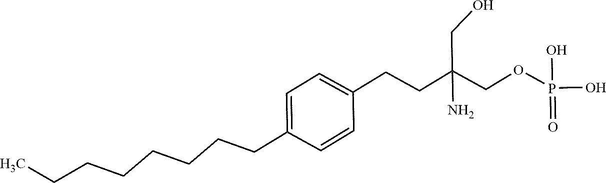 [SMILES: CCCCCCCCc1ccc(CCC(N)(CO)COP(=O)(O)O)cc1]

or a derivative thereof.

In some embodiments, the disclosure provides methods for preventing and/or treating myopia by administering to a subject a therapeutically effective amount of fingolimod phosphate or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a fingolimod phosphate or a derivative thereof, in a form of oral composition, extended drug release formulation or composition, extended drug delivery by contact lenses, or eye drops during a susceptible period for myopia development.

In further embodiments, the active drug compound is a sphingosine-1-phosphate receptor modulator.

In some embodiments, the sphingosine-1-phosphate receptor modulator includes but is not limited to, ONO-4641, ozanimod, siponimod, ponesimod, and derivatives thereof.

Thus, in further embodiments, the disclosure provides methods for preventing and/or treating myopia by administering to a subject a therapeutically effective amount of a sphingosine-1-phosphate receptor modulator, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a sphingosine-1-phosphate receptor modulator, in a form of oral composition, extended drug release formulation or composition, extended drug delivery by contact lenses, or eye drops during a susceptible period for myopia development.

In a further embodiment, the active drug compound is a berberidaceaen alkaloid, berberine chloride, having the structure:

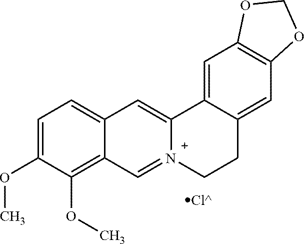

or a derivative thereof.

In some embodiments, the disclosure provides methods for preventing and/or treating myopia by administering to a subject a therapeutically effective amount of berberine chloride or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a berberine chloride or a derivative thereof, in a form of oral composition, extended drug release formulation or composition, extended drug delivery by contact lenses, or eye drops during a susceptible period for myopia development.

In further embodiments, the active drug compound is a berberidaceaen alkaloid.

Thus, in further embodiments, the disclosure provides methods for preventing and/or treating myopia by administering to a subject a therapeutically effective amount of a berberidaceaen alkaloid, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a berberidaceaen alkaloid, in a form of oral composition, extended drug release formulation or composition, extended drug delivery by contact lenses, or eye drops during a susceptible period for myopia development.

In a further embodiment, the active drug compound is a selective neuronal nitric oxide synthase (nNOS) inhibitor, S-methyl-L-thiocitrulline hydrochloride, having the structure:

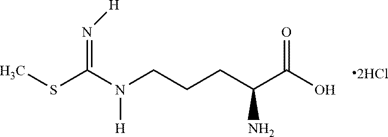

or a derivative thereof.

In some embodiments, the disclosure provides methods for preventing and/or treating myopia by administering to a subject a therapeutically effective amount of S-methyl-L-thiocitrulline hydrochloride or a derivative thereof, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of S-methyl-L-thiocitrulline hydrochloride or a derivative thereof, in a form of oral composition, extended drug release formulation or composition, extended drug delivery by contact lenses, or eye drops during a susceptible period for myopia development.

In further embodiments, the active drug compound is a selective neuronal nitric oxide synthase (nNOS) inhibitor.

In some embodiments, the selective nNOS inhibitor includes but is not limited to, N^{ω}-propyl-L-arginine hydrochloride and derivatives thereof.

Thus, in further embodiments, the disclosure provides methods for preventing and/or treating myopia by administering to a subject a therapeutically effective amount of a selective nNOS inhibitor, in a form of oral composition, extended drug release formulation or composition, extended drug delivery by contact lenses, or eye drops during a susceptible period for myopia development.

In some embodiments, the disclosure provides methods for preventing and/or treating myopia by administering to a subject a repeating dose of a therapeutically effective amount of a selective nNOS inhibitor, in a form of oral composition, extended drug release formulation or composition, extended drug delivery by contact lenses, or eye drops during a susceptible period for myopia development.

In some embodiments, the composition is administered to the subject once a day. In some embodiments, the composition is administered once a week. In some embodiments, the composition is administered twice a week. In some embodiments, the composition is administered three times a week. In some embodiments, the composition is administered to the subject continuously or intermittently for about 5 years to about 10 years.

In some embodiments, the subject is a young adult, i.e., under 30 years of age. In some embodiments, the subject is a child, i.e., under the age of 18. In some embodiments, the subject is about 4 years of age to about 30 years of age. In some embodiments, the subject is about 6 years of age to about 20 years of age. In some embodiments, the subject is about 8 years of age to about 15 years of age. In some embodiments, the subject is about 10 years of age to about 12 years of age.

In some embodiments, the subject has myopia. In some embodiments, the subject is at risk for myopia. In some embodiments, the subject is susceptible to myopia.

In some embodiments, the subject is monitored for suppression of myopia and the therapeutically effective amount and/or frequency of administration of the drug compound is adjusted depending on the degree of suppression. Suppression of myopia may be monitored using methods known in the art.

A further embodiment of the present disclosure are kits comprising compositions and agents for practicing the disclosed methods.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Experimentally induced myopia in mice has features of human myopia. FIG. 1A shows a mouse induced to have myopia with -25 D lenses. FIG. 1B is a graph show the statistically significant myopic shift in refraction observed in the eyes of the mice treated with -25 D lenses for 21 days. FIG. 1C shows that the lens-induced myopia in mice is due to a statistically significant increase in the vitreous chamber depth, as in human myopia. FIG. 1D shows the power simulations demonstrating the relationship between statistical power and a number of animals for induced myopia experiments. ACD, anterior chamber depth; CRC, corneal radius of curvature; LT, lens thickness; VCD, vitreous chamber depth; OD, right (myopic) eye; OS, left (control) eye. Error bars, SD. P, significance value.

FIG. 2 shows that systemic administration of 5 mg/kg levocabastine hydrochloride completely suppressed development of myopia in mice with experimentally induced myopia.

FIG. 3 shows that systemic administration of 1 mg/kg fenoterol hydrobromide suppressed development of myopia in mice with experimentally induced myopia by approximately 90%.

FIG. 4 shows that systemic administration of 1 mg/kg trichostatin A suppressed development of myopia in mice with experimentally induced myopia by more than 100%.

FIG. 5 shows that systemic administration of 0.3 mg/kg fingolimod phosphate completely suppressed development of myopia in mice with experimentally induced myopia.

FIG. 6 shows that systemic administration of 20 mg/kg berberine chloride suppressed development of myopia in mice with experimentally induced myopia by approximately 50%.

FIG. 7 shows that systemic administration of 10 mg/kg S-methyl-L-thiocitrulline hydrochloride suppressed development of myopia in mice with experimentally induced myopia by approximately 97%.

### DETAILED DESCRIPTION

### Definitions

The following definitions and explanations are meant and intended to be controlling in any construction unless clearly and unambiguously modified in the following examples or when application of the meaning renders any construction meaningless or essentially meaningless. In cases where the construction of the term would render it meaningless or essentially meaningless, the definition should be taken from Webster’s Dictionary or a dictionary known to those of skill in the art, such as the Oxford Dictionary of Biochemistry and Molecular Biology or similar.

The contents of any patents, patent applications, and references cited throughout this specification are hereby incorporated by reference in their entireties.

As used herein and unless otherwise indicated, the terms “a” and “an” are taken to mean “one”, “at least one” or “one or more”. Unless otherwise required by context, singular terms used herein shall include pluralities and plural terms shall include the singular.

The term “myopia” or “myopic” shall mean eye disease condition in which the posterior segment of the eye is too large for the optical power of the eye and the focal point is located in front of the retina; thus, producing blurred distant vision.

The term “hyperopia” or “hyperopic” shall mean eye condition in which the posterior segment of the eye is too small for the optical power of the eye and the focal point is located behind the retina; thus, producing blurred near vision.

The term “negative lens” shall mean a lens which shifts focal point of the eye towards the back of the eye; thus, rendering the eye hyperopic.

The term “genetic network” shall mean a network of interconnected genes which regulate a physiological or biological process.

The term “differentially expressed” shall mean changes in gene expression level induced by environmental factors, changes in genetic background, or other internal or external insult or influence.

The term “experimentally induced myopia” is used here to describe myopia induced in animal models by experimental manipulations, such as the application of negative lenses over the eye.

The term “whole-genome gene expression profiling” refers to a method of analyzing differential gene expression at the level of the entire genome; thus, providing information about expression of all genes encoded by the genome.

The term “gene-based genome-wide association study” refers to a genetic study which analyzes statistical associations between genetic variations in the genome and a disease at the level of specific genes, found previously to be involved in a disease process by other experimental approaches such as whole-genome gene expression profiling.

The term “positive optical defocus’ shall mean the condition when focal point of the eye is located in front of the retina.

The term “negative optical defocus” shall mean the condition when focal point of the eye is located behind the retina.

The term “derivative” refers to structural analog of a compound that is derived from a compound by a chemical reaction. A structural analog is a compound having a structure similar to that of another compound but differing from it in respect to a certain component. It can differ in one or more atoms, functional groups, or substructures, which are replaced with other atoms, groups, or substructures. A structural analog can also differ from another compound in one or more atoms, functional groups, or substructures, which are added to or subtracted from another compound. A structural analog can be imagined to be formed by those skilled in art, at least theoretically, from the other compound.

The term “subject” as used in this application means a human subject. In some embodiments of the present invention, the “subject” has myopia, is at risk for myopia or is susceptible to myopia.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known to be, or suspected of, suffering from myopia.

A subject in need of treatment would be one that has already developed the disease or condition. A subject in need of prevention would be one with risk factors of the disease or condition.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

### Identifying Anti-Myopia Drugs Using a Pharmacogenomic Pipeline

Shown herein is the results of the use of a pharmacogenomic pipeline developed by the inventors for the identification of drug compounds capable of suppressing myopia development.⁸⁰ A systems genetics approach was used to identify genes, genetic networks, and signaling pathways underlying refractive eye development and the development of myopia. The systems genetics approach comprised identification of genes differentially expressed in the eyes of animals with experimentally induced myopia using whole-genome gene expression profiling and identification of genes linked to myopia in humans using gene-based genome-wide association studies. One of the inventors’ studies found that signaling pathways underlying eye’s responses to positive optical defocus (which suppresses myopia) and negative optical defocus (which promotes myopia development) propagate via two largely distinct signaling cascades, described in U.S. Provisional Application No. 62/730,301.

The inventors extended this observation to several vertebrate species and demonstrated that signaling cascades underlying myopia development are highly evolutionarily conserved across vertebrate species, including humans. The inventors then used their vast myopia-associated gene dataset (which included over 3,500 genes) described in Tkatchenko et al. 2019,⁸¹ and computational tools to reconstruct the genetic networks that control myopia development and to identify drug compounds, which can suppress signaling pathways that promote myopia development and stimulate the pathways that inhibit the development of myopia.

A total of 138 drug compounds with anti-myopic potential were identified. Using the gene pathways and z-scores, these drug compounds were assigned to top 10, top 20, top 40, top 80, and low priority categories based on their predicted potential to suppress myopia and known or predicted side effects. These drug compounds were then tested on a mouse model of myopia (Example 1).

### Methods and Compositions for the Prevention and/or Treatment of Myopia Using Levocabastine, a Selective Histamine H1-Receptor Antagonist, and Derivatives Thereof

The disclosure provides in some aspects methods of preventing and/or treating myopia comprising administering to a subject in need thereof a therapeutically effective amount of levocabastine hydrochloride or a derivative thereof.

In certain embodiments, the levocabastine hydrochloride or derivative is administered systemically. In certain embodiments, the levocabastine hydrochloride or derivative is administered orally. In certain embodiments, the levocabastine hydrochloride or derivative is administered locally. In some embodiments, the levocabastine hydrochloride or derivative is administered directly to or into the eye. In some embodiments, the levocabastine hydrochloride or derivative is administered via injection. In other embodiments, the levocabastine hydrochloride or derivative is administered as extended drug release formulations or compositions, extended drug delivery by contact lenses, or eye drops.

In certain embodiments, the levocabastine hydrochloride is used directly as the active ingredient in the drug. In other embodiments, the levocabastine hydrochloride can be chemically modified to improve its efficacy, reduce side effects, improve penetration through ocular tissues, increase stability, or improve bioavailability.

In certain embodiments, the levocabastine hydrochloride (or its derivative) is a sole component of the drug. In other embodiments, the methods and compositions described herein comprise the use of pharmaceutical formulations comprising the levocabastine hydrochloride (or its derivative).

The term “pharmaceutical formulation” refers to preparations, which include the levocabastine hydrochloride (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

Levocabastine hydrochloride (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

Levocabastine hydrochloride (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

Levocabastine hydrochloride (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the levocabastine hydrochloride (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The levocabastine hydrochloride described herein can be co-administered with other agents including additional agents for the prevention and/or treatment of myopia. The co-administration of agents can be by any administration described herein. Moreover, the additional agent can be in the same composition as the levocabastine hydrochloride. The additional agent can be in a separate composition from the levocabastine hydrochloride. The administration of more than one composition can be simultaneous, concurrently or sequentially.

The disclosure further provides in some aspects methods of preventing and/or treating myopia comprising administering to a subject in need thereof a therapeutically effective amount of a selective histamine H1-receptor antagonist.

In some embodiments, the selective histamine H1-receptor antagonist includes but is not limited to azelastine, alcaftadine, antazoline, carbinoxamine, epinastine, olopatadine, ketotifen, or derivatives thereof.

In certain embodiments, the selective histamine H1-receptor antagonist is administered systemically. In certain embodiments, the selective histamine H1-receptor antagonist is administered orally. In certain embodiments, the selective histamine H1-receptor antagonist is administered locally. In some embodiments, the selective histamine H1-receptor antagonist is administered directly to or into the eye. In some embodiments, the selective histamine H1-receptor antagonist is administered via injection. In other embodiments, the selective histamine H1-receptor antagonist is administered as extended drug release formulations or compositions, extended drug delivery by contact lenses, or eye drops.

In further embodiments, the selective histamine H1-receptor antagonist is used directly as the active ingredient in the drug. In other embodiments, the selective histamine H1-receptor antagonist can be chemically modified to improve its efficacy, reduce side effects, improve penetration through ocular tissues, increase stability, or improve bioavailability.

In certain embodiments, the selective histamine H1-receptor antagonist is a sole component of the drug. In other embodiments, the methods and compositions described herein comprise the use of pharmaceutical formulations comprising the selective histamine H1-receptor antagonist.

The term “pharmaceutical formulation” refers to preparations, which include the selective histamine H1-receptor antagonist and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The selective histamine H1-receptor antagonist is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The selective histamine H1-receptor antagonist is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The selective histamine H1-receptor antagonist is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the selective histamine H1-receptor antagonist is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The selective histamine H1-receptor antagonist described herein can be co-administered with other agents including additional agents for the suppression, prevention and/or treatment of myopia. The co-administration of agents can be by any administration described herein. Moreover, the additional agent can be in the same composition as the selective histamine H1-receptor antagonist. The additional agent can be in a separate composition from the selective histamine H1-receptor antagonist. The administration of more than one composition can be simultaneous, concurrently or sequentially.

### Methods and Compositions for the Prevention and/or Treatment of Myopia Using Fenoterol Hydrobromide, a β2-adrenergic Receptor Agonist, and Derivatives Thereof

The disclosure provides in some aspects methods of preventing and/or treating myopia comprising administering to a subject in need thereof a therapeutically effective amount of fenoterol hydrobromide or a derivative thereof.

In certain embodiments, the fenoterol hydrobromide or derivative is administered systemically. In certain embodiments, the fenoterol hydrobromide or derivative is administered orally. In certain embodiments, the fenoterol hydrobromide or derivative is administered locally. In some embodiments, the fenoterol hydrobromide or derivative is administered directly to or into the eye. In some embodiments, the fenoterol hydrobromide or derivative is administered via injection. In other embodiments, the fenoterol hydrobromide or derivative is administered as extended drug release formulations or compositions, extended drug delivery by contact lenses, or eye drops.

In certain embodiments, the fenoterol hydrobromide is used directly as the active ingredient in the drug. In other embodiments, the fenoterol hydrobromide can be chemically modified to improve its efficacy, reduce side effects, improve penetration through ocular tissues, increase stability, or improve bioavailability.

In certain embodiments, the fenoterol hydrobromide (or its derivative) is a sole component of the drug. In other embodiments, the methods and compositions described herein comprise the use of pharmaceutical formulations comprising the fenoterol hydrobromide (or its derivative).

The term “pharmaceutical formulation” refers to preparations, which include the fenoterol hydrobromide (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

Fenoterol hydrobromide (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

Fenoterol hydrobromide (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

Fenoterol hydrobromide (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the fenoterol hydrobromide (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The fenoterol hydrobromide described herein can be co-administered with other agents including additional agents for the prevention and/or treatment of myopia. The co-administration of agents can be by any administration described herein. Moreover, the additional agent can be in the same composition as the fenoterol hydrobromide. The additional agent can be in a separate composition from the fenoterol hydrobromide. The administration of more than one composition can be simultaneous, concurrently or sequentially.

The disclosure further provides in some aspects methods of preventing and/or treating myopia comprising administering to a subject in need thereof a therapeutically effective amount of a β2-adrenergic receptor agonist.

In some embodiments, the β2-adrenergic receptor agonist includes but is not limited to, bitolterol, isoprenaline, levosalbutamol, levalbuterol, orciprenaline, metaproterenol, pirbuterol, procaterol, ritodrine, salbutamol, albuterol, terbutaline, arformoterol, bambuterol, clenbuterol, formoterol, salmeterol, or derivatives thereof.

In certain embodiments, the β2-adrenergic receptor agonist is administered systemically. In certain embodiments, the β2-adrenergic receptor agonist is administered orally. In certain embodiments, the β2-adrenergic receptor agonist is administered locally. In some embodiments, the β2-adrenergic receptor agonist is administered directly to or into the eye. In some embodiments, the β2-adrenergic receptor agonist is administered via injection. In other embodiments, the β2-adrenergic receptor agonist is administered as extended drug release formulations or compositions, extended drug delivery by contact lenses, or eye drops.

In further embodiments, the β2-adrenergic receptor agonist is used directly as the active ingredient in the drug. In other embodiments, the β2-adrenergic receptor agonist can be chemically modified to improve its efficacy, reduce side effects, improve penetration through ocular tissues, increase stability, or improve bioavailability.

In certain embodiments, the β2-adrenergic receptor agonist is a sole component of the drug. In other embodiments, the methods and compositions described herein comprise the use of pharmaceutical formulations comprising the β2-adrenergic receptor agonist.

The term “pharmaceutical formulation” refers to preparations, which include the β2-adrenergic receptor agonist and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The β2-adrenergic receptor agonist is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The β2-adrenergic receptor agonist is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The β2-adrenergic receptor agonist is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the β2-adrenergic receptor agonist is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The β2-adrenergic receptor agonist described herein can be co-administered with other agents including additional agents for the suppression, prevention and/or treatment of myopia. The co-administration of agents can be by any administration described herein. Moreover, the additional agent can be in the same composition as the β2-adrenergic receptor agonist. The additional agent can be in a separate composition from the β2-adrenergic receptor agonist. The administration of more than one composition can be simultaneous, concurrently or sequentially.

### Methods and Compositions for the Prevention and/or Treatment of Myopia Using Trichostatin A, a Histone Deacetylase (HDAC) Inhibitor, and Derivatives Thereof

The disclosure provides in some aspects methods of preventing and/or treating myopia comprising administering to a subject in need thereof a therapeutically effective amount of trichostatin A or a derivative thereof.

In certain embodiments, the trichostatin A or derivative is administered systemically. In certain embodiments, the trichostatin A or derivative is administered orally. In certain embodiments, the trichostatin A or derivative is administered locally. In some embodiments, the trichostatin A or derivative is administered directly to or into the eye. In some embodiments, the trichostatin A or derivative is administered via injection. In other embodiments, the trichostatin A or derivative is administered as extended drug release formulations or compositions, extended drug delivery by contact lenses, or eye drops.

In certain embodiments, the trichostatin A is used directly as the active ingredient in the drug. In other embodiments, the trichostatin A can be chemically modified to improve its efficacy, reduce side effects, improve penetration through ocular tissues, increase stability, or improve bioavailability.

In certain embodiments, the trichostatin A (or its derivative) is a sole component of the drug. In other embodiments, the methods and compositions described herein comprise the use of pharmaceutical formulations comprising the trichostatin A (or its derivative).

The term “pharmaceutical formulation” refers to preparations, which include the trichostatin A (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

Trichostatin A (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

Trichostatin A (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

Trichostatin A (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the trichostatin A (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The trichostatin A described herein can be co-administered with other agents including additional agents for the prevention and/or treatment of myopia. The co-administration of agents can be by any administration described herein. Moreover, the additional agent can be in the same composition as the trichostatin A. The additional agent can be in a separate composition from the trichostatin A. The administration of more than one composition can be simultaneous, concurrently or sequentially.

The disclosure further provides in some aspects methods of preventing and/or treating myopia comprising administering to a subject in need thereof a therapeutically effective amount of an HDAC inhibitor.

In some embodiments, the HDAC inhibitor includes but is not limited to, vorinostat, belinostat, panobinostat, entinostat, and mocetinostat, and derivatives thereof.

In certain embodiments, the HDAC inhibitor is administered systemically. In certain embodiments, the HDAC inhibitor is administered orally. In certain embodiments, the HDAC inhibitor is administered locally. In some embodiments, the HDAC inhibitor is administered directly to or into the eye. In some embodiments, the HDAC inhibitor is administered via injection. In other embodiments, the HDAC inhibitor is administered as extended drug release formulations or compositions, extended drug delivery by contact lenses, or eye drops.

In further embodiments, the HDAC inhibitor is used directly as the active ingredient in the drug. In other embodiments, the HDAC inhibitor can be chemically modified to improve its efficacy, reduce side effects, improve penetration through ocular tissues, increase stability, or improve bioavailability.

In certain embodiments, the HDAC inhibitor is a sole component of the drug. In other embodiments, the methods and compositions described herein comprise the use of pharmaceutical formulations comprising the HDAC inhibitor.

The term “pharmaceutical formulation” refers to preparations, which include the HDAC inhibitor and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The HDAC inhibitor is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The HDAC inhibitor is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The HDAC inhibitor is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the HDAC inhibitor is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The HDAC inhibitor described herein can be co-administered with other agents including additional agents for the suppression, prevention and/or treatment of myopia. The co-administration of agents can be by any administration described herein. Moreover, the additional agent can be in the same composition as the HDAC inhibitor. The additional agent can be in a separate composition from the HDAC inhibitor. The administration of more than one composition can be simultaneous, concurrently or sequentially.

### Methods and Compositions for the Prevention and/or Treatment of Myopia Using Fingolimod, a Sphingosine-1-Phosphate Receptor Modulator, and Derivatives Thereof

The disclosure provides in some aspects methods of preventing and/or treating myopia comprising administering to a subject in need thereof a therapeutically effective amount of fingolimod (FTY720) phosphate or a derivative thereof.

In certain embodiments, the fingolimod phosphate or derivative is administered systemically. In certain embodiments, the fingolimod phosphate or derivative is administered orally. In certain embodiments, the fingolimod phosphate or derivative is administered locally. In some embodiments, the fingolimod phosphate or derivative is administered directly to or into the eye. In some embodiments, the fingolimod phosphate or derivative is administered via injection. In other embodiments, the fingolimod phosphate or derivative is administered as extended drug release formulations or compositions, extended drug delivery by contact lenses, or eye drops.

In certain embodiments, the fingolimod phosphate is used directly as the active ingredient in the drug. In other embodiments, the fingolimod phosphate can be chemically modified to improve its efficacy, reduce side effects, improve penetration through ocular tissues, increase stability, or improve bioavailability.

In certain embodiments, the fingolimod phosphate (or its derivative) is a sole component of the drug. In other embodiments, the methods and compositions described herein comprise the use of pharmaceutical formulations comprising the fingolimod phosphate (or its derivative).

The term “pharmaceutical formulation” refers to preparations, which include the fingolimod phosphate (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

Fingolimod phosphate (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

Fingolimod phosphate (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

Fingolimod phosphate (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the fingolimod phosphate (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The fingolimod phosphate described herein can be co-administered with other agents including additional agents for the prevention and/or treatment of myopia. The co-administration of agents can be by any administration described herein. Moreover, the additional agent can be in the same composition as the fingolimod phosphate. The additional agent can be in a separate composition from the fingolimod phosphate. The administration of more than one composition can be simultaneous, concurrently or sequentially.

The disclosure further provides in some aspects methods of preventing and/or treating myopia comprising administering to a subject in need thereof a therapeutically effective amount of a sphingosine-1-phosphate receptor modulator.

In some embodiments, the sphingosine-1-phosphate receptor modulator includes but is not limited to, ONO-4641, ozanimod, siponimod, ponesimod, or derivatives thereof.

In certain embodiments, the sphingosine-1-phosphate receptor modulator is administered systemically. In certain embodiments, the sphingosine-1-phosphate receptor modulator is administered orally. In certain embodiments, the sphingosine-1-phosphate receptor modulator is administered locally. In some embodiments, the sphingosine-1-phosphate receptor modulator is administered directly to or into the eye. In some embodiments, the sphingosine-1-phosphate receptor modulator is administered via injection. In other embodiments, the sphingosine-1-phosphate receptor modulator is administered as extended drug release formulations or compositions, extended drug delivery by contact lenses, or eye drops.

In further embodiments, the sphingosine-1-phosphate receptor modulator is used directly as the active ingredient in the drug. In other embodiments, the sphingosine-1-phosphate receptor modulator can be chemically modified to improve its efficacy, reduce side effects, improve penetration through ocular tissues, increase stability, or improve bioavailability.

In certain embodiments, the sphingosine-1-phosphate receptor modulator is a sole component of the drug. In other embodiments, the methods and compositions described herein comprise the use of pharmaceutical formulations comprising the sphingosine-1-phosphate receptor modulator.

The term “pharmaceutical formulation” refers to preparations, which include the sphingosine-1-phosphate receptor modulator and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The sphingosine-1-phosphate receptor modulator is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The sphingosine-1-phosphate receptor modulator is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The sphingosine-1-phosphate receptor modulator is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the sphingosine-1-phosphate receptor modulator is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The sphingosine-1-phosphate receptor modulator described herein can be co-administered with other agents including additional agents for the suppression, prevention and/or treatment of myopia. The co-administration of agents can be by any administration described herein. Moreover, the additional agent can be in the same composition as the sphingosine-1-phosphate receptor modulator. The additional agent can be in a separate composition from the sphingosine-1-phosphate receptor modulator. The administration of more than one composition can be simultaneous, concurrently or sequentially.

### Methods and Compositions for the Prevention and/or Treatment of Myopia Using Berberine Chloride, a Berberidaceaen Alkaloid, and Derivatives Thereof

The disclosure provides in some aspects methods of preventing and/or treating myopia comprising administering to a subject in need thereof a therapeutically effective amount of berberine chloride or a derivative thereof.

In certain embodiments, the berberine chloride or derivative is administered systemically. In certain embodiments, the berberine chloride or derivative is administered orally. In certain embodiments, the berberine chloride or derivative is administered locally. In some embodiments, the berberine chloride or derivative is administered directly to or into the eye. In some embodiments, the berberine chloride or derivative is administered via injection. In other embodiments, the berberine chloride or derivative is administered as extended drug release formulations or compositions, extended drug delivery by contact lenses, or eye drops.

In certain embodiments, the berberine chloride is used directly as the active ingredient in the drug. In other embodiments, the berberine chloride can be chemically modified to improve its efficacy, reduce side effects, improve penetration through ocular tissues, increase stability, or improve bioavailability.

In certain embodiments, the berberine chloride (or its derivative) is a sole component of the drug. In other embodiments, the methods and compositions described herein comprise the use of pharmaceutical formulations comprising the berberine chloride (or its derivative).

The term “pharmaceutical formulation” refers to preparations, which include the berberine chloride (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

Berberine chloride (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

Berberine chloride (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

Berberine chloride (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the berberine chloride (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The berberine chloride described herein can be co-administered with other agents including additional agents for the prevention and/or treatment of myopia. The co-administration of agents can be by any administration described herein. Moreover, the additional agent can be in the same composition as the berberine chloride. The additional agent can be in a separate composition from the berberine chloride. The administration of more than one composition can be simultaneous, concurrently or sequentially.

The disclosure further provides in some aspects methods of preventing and/or treating myopia comprising administering to a subject in need thereof a therapeutically effective amount of a berberidaceaen alkaloid.

In certain embodiments, the berberidaceaen alkaloid is administered systemically. In certain embodiments, the berberidaceaen alkaloid is administered orally. In certain embodiments, the berberidaceaen alkaloid is administered locally. In some embodiments, the berberidaceaen alkaloid is administered directly to or into the eye. In some embodiments, the berberidaceaen alkaloid is administered via injection. In other embodiments, the berberidaceaen alkaloid is administered as extended drug release formulations or compositions, extended drug delivery by contact lenses, or eye drops.

In further embodiments, the berberidaceaen alkaloid is used directly as the active ingredient in the drug. In other embodiments, the berberidaceaen alkaloid can be chemically modified to improve its efficacy, reduce side effects, improve penetration through ocular tissues, increase stability, or improve bioavailability.

In certain embodiments, the berberidaceaen alkaloid is a sole component of the drug. In other embodiments, the methods and compositions described herein comprise the use of pharmaceutical formulations comprising the berberidaceaen alkaloid.

The term “pharmaceutical formulation” refers to preparations, which include the berberidaceaen alkaloid and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The berberidaceaen alkaloid is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The berberidaceaen alkaloid is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The berberidaceaen alkaloid is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the berberidaceaen alkaloid is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The berberidaceaen alkaloid described herein can be co-administered with other agents including additional agents for the suppression, prevention and/or treatment of myopia. The co-administration of agents can be by any administration described herein. Moreover, the additional agent can be in the same composition as the berberidaceaen alkaloid. The additional agent can be in a separate composition from the berberidaceaen alkaloid. The administration of more than one composition can be simultaneous, concurrently or sequentially.

### Methods and Compositions for the Prevention And/or Treatment of Myopia Using S-Methyl-L-Thiocitrulline Hydrochloride, a Selective Neuronal Nitric Oxide Synthase (nNOS) Inhibitor, and Derivatives Thereof

The disclosure provides in some aspects methods of preventing and/or treating myopia comprising administering to a subject in need thereof a therapeutically effective amount of S-methyl-L-thiocitrulline hydrochloride or a derivative thereof.

In certain embodiments, the S-methyl-L-thiocitrulline hydrochloride or derivative is administered systemically. In certain embodiments, the S-methyl-L-thiocitrulline hydrochloride or derivative is administered orally. In certain embodiments, the S-methyl-L-thiocitrulline hydrochloride or derivative is administered locally. In some embodiments, the S-methyl-L-thiocitrulline hydrochloride or derivative is administered directly to or into the eye. In some embodiments, the S-methyl-L-thiocitrulline hydrochloride or derivative is administered via injection. In other embodiments, the S-methyl-L-thiocitrulline hydrochloride or derivative is administered as extended drug release formulations or compositions, extended drug delivery by contact lenses, or eye drops.

In certain embodiments, the S-methyl-L-thiocitrulline hydrochloride is used directly as the active ingredient in the drug. In other embodiments, the S-methyl-L-thiocitrulline hydrochloride can be chemically modified to improve its efficacy, reduce side effects, improve penetration through ocular tissues, increase stability, or improve bioavailability.

In certain embodiments, the S-methyl-L-thiocitrulline hydrochloride (or its derivative) is a sole component of the drug. In other embodiments, the methods and compositions described herein comprise the use of pharmaceutical formulations comprising the S-methyl-L-thiocitrulline hydrochloride (or its derivative).

The term “pharmaceutical formulation” refers to preparations, which include the S-methyl-L-thiocitrulline hydrochloride (or its derivative) and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

S-methyl-L-thiocitrulline hydrochloride (or its derivative) is considered to retain its physical properties in a pharmaceutical formulation if it meets defined specifications for degradation, and/or aggregation, and/or precipitation upon visual examination of color and/or clarity, or as measured by light scattering or other suitable art recognized methods.

S-methyl-L-thiocitrulline hydrochloride (or its derivative) is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

S-methyl-L-thiocitrulline hydrochloride (or its derivative) is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the S-methyl-L-thiocitrulline hydrochloride (or its derivative) is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The S-methyl-L-thiocitrulline hydrochloride described herein can be co-administered with other agents including additional agents for the prevention and/or treatment of myopia. The co-administration of agents can be by any administration described herein. Moreover, the additional agent can be in the same composition as the S-methyl-L-thiocitrulline hydrochloride. The additional agent can be in a separate composition from the S-methyl-L-thiocitrulline hydrochloride. The administration of more than one composition can be simultaneous, concurrently or sequentially.

The disclosure further provides in some aspects methods of preventing and/or treating myopia comprising administering to a subject in need thereof a therapeutically effective amount of a selective neuronal nitric oxide synthase (nNOS) inhibitor.

In some embodiments, the selective neuronal nitric oxide synthase (nNOS) inhibitor includes but is not limited to, N^{ω}-propyl-L-arginine hydrochloride or derivatives thereof.

In certain embodiments, the selective nNOS inhibitor is administered systemically. In certain embodiments, the selective nNOS inhibitor is administered orally. In certain embodiments, the selective nNOS inhibitor is administered locally. In some embodiments, the selective nNOS inhibitor is administered directly to or into the eye. In some embodiments, the selective nNOS inhibitor is administered via injection. In other embodiments, the selective nNOS inhibitor is administered as extended drug release formulations or compositions, extended drug delivery by contact lenses, or eye drops.

In further embodiments, the selective nNOS inhibitor is used directly as the active ingredient in the drug. In other embodiments, the selective nNOS inhibitor can be chemically modified to improve its efficacy, reduce side effects, improve penetration through ocular tissues, increase stability, or improve bioavailability.

In certain embodiments, the selective neuronal nNOS inhibitor is a sole component of the drug. In other embodiments, the methods and compositions described herein comprise the use of pharmaceutical formulations comprising the selective nNOS inhibitor.

The term “pharmaceutical formulation” refers to preparations, which include the selective nNOS inhibitor and additional ingredients, such as other drugs capable of suppressing myopia or excipients (vehicles, additives, preservatives, buffers), which can reasonably be administered to a subject to improve the efficacy of the active ingredient(s) or increase stability of the active ingredient(s). A formulation is stable if the active ingredient(s) essentially retain their physical properties, and/or chemical properties, and/or biological activity at room temperature (15-30° C.) for at least a week, or at 2-8° C. for 3 months to 1 year.

The selective nNOS inhibitor is considered to retain its physical properties in a pharmaceutical formulation if it meets defined specifications for degradation, and/or aggregation, and/or precipitation upon visual examination of color and/or clarity, or as measured by light scattering or other suitable art recognized methods.

The selective nNOS inhibitor is considered to retain its chemical stability in a pharmaceutical formulation if the active ingredient content within about 90% of the amount at the time the pharmaceutical formulation was prepared. Some types of chemical degradation include oxidation and hydrolysis, which can be evaluated, for example, by LC-MS/MS-based methods.

The selective nNOS inhibitor is considered to retain its biological stability in a pharmaceutical formulation if the active ingredient at a given time is within about 90% of the biological activity exhibited at the time the pharmaceutical formulation was prepared as determined by in vivo testing, for example.

In the context of the present disclosure, the therapeutically effective dose of the selective nNOS inhibitor is the amount sufficient to at least partially prevent and/or treat myopia. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can significantly slow the progression of myopia in a subject suffering from the disease. The dose and frequency of drug administration effective for this use will depend on the severity of the disease (i.e., low progressing versus high progressing myopia), type of myopia (i.e., syndromic myopia versus common myopia), subject age, body mass of the subject, and route of administration among other factors. The dose and frequency of the drug administration can be adjusted using well understood and commonly used state of art in optometric and ophthalmologic practices.

The selective nNOS inhibitor described herein can be co-administered with other agents including additional agents for the suppression, prevention and/or treatment of myopia. The co-administration of agents can be by any administration described herein. Moreover, the additional agent can be in the same composition as the selective nNOS inhibitor. The additional agent can be in a separate composition from the selective nNOS inhibitor. The administration of more than one composition can be simultaneous, concurrently or sequentially.

Oral compositions of the drug can be in a form of capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

It should be understood that, in addition to the ingredients particularly mentioned above, the compositions may include other agents conventional in the art having regard to the type of formulation in question, for example those suitable for oral administration may include flavoring agents.

Extended drug release formulations or compositions can be in a form of a nanosponge, patch, gel or other device capable of gradual release of the drug over extended period of time, which is injected in the anterior or posterior segment of the eye or administered or applied to the anterior or posterior surfaces of the eye.

Extended drug delivery by contact lenses can be in a form of plano contact lens, single-vision corrective contact lens, or multi-focal contact lens, in which either internal surface of the lens is coated with the drug, or the entire volume of the lens is loaded with the drug.

Eye drops can be in a form of traditional eye drops well-known and commonly used by those skilled in the art, or in a form of a micro-dosing device which delivers a strictly controlled amount of the drug to the eye.

In some embodiments, the composition is administered to the subject once a day. In some embodiments, the composition is administered once a week. In some embodiments, the composition is administered twice a week. In some embodiments, the composition is administered three times a week. In some embodiments, the composition is administered to the subject continuously or intermittently for about 5 years to about 10 years.

In some embodiments, the composition is administered more than once.

Treatment using the present methods and compositions can continue as long as needed.

In one embodiment, the efficacy of the treatment in a subject with myopia is evaluated every 3-6 months and the dose and/or frequency of drug administration is adjusted depending on the degree of myopia suppression. The treatment is discontinued once the subject does not exhibit any further progression of myopia, which can be evaluated by temporarily discontinuing the treatment and measuring changes in refractive error over 1-6 months using well understood state of art in optometric and ophthalmologic practices.

In some embodiments, the subject is a child, i.e., under 18 years of age. In some embodiments, the subject is a young adult, i.e., under 30 years of age. In some embodiments, the subject is about 4 years of age to about 30 years of age. In some embodiments, the subject is about 6 years of age to about 20 years of age. In some embodiments, the subject is about 8 years of age to about 15 years of age. In some embodiments, the subject is about 10 years of age to about 12 years of age.

In some embodiments, the subject has myopia. In some embodiments, the subject is at risk for myopia. In some embodiments, the subject is susceptible to myopia.

Risk factors for myopia can include but are not limited to having one or more parents with myopia.

### Kits

Also within the scope of the present disclosure are kits for practicing the disclosed methods.

In some embodiments, the kit can comprise instructions for use in any of the methods described herein. The included instructions can comprise a description of administration of the agents to a subject to achieve the intended activity in a subject. The kit may further comprise a description of selecting a subject suitable for treatment based on identifying whether the subject is in need of the treatment.

The instructions relating to the use of the drugs described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### EXAMPLES

The following examples are included to demonstrate preferred embodiments of the invention. In light of the present disclosure, it should be appreciated by those of skill in the art that many changes can be made in the specific embodiments which are disclosed and still obtain a like or similar result without departing from the spirit and scope of the invention.

### Example 1. Myopia Can Be Induced in Mammals Using Negative Spectacle Lenses

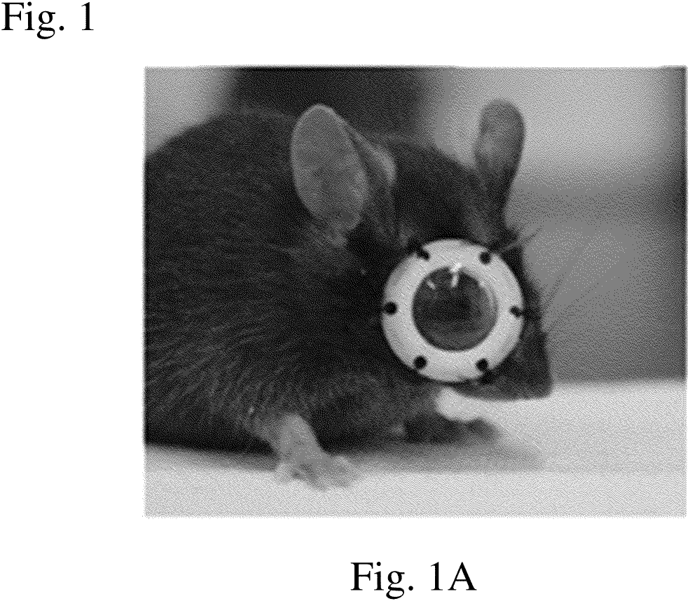

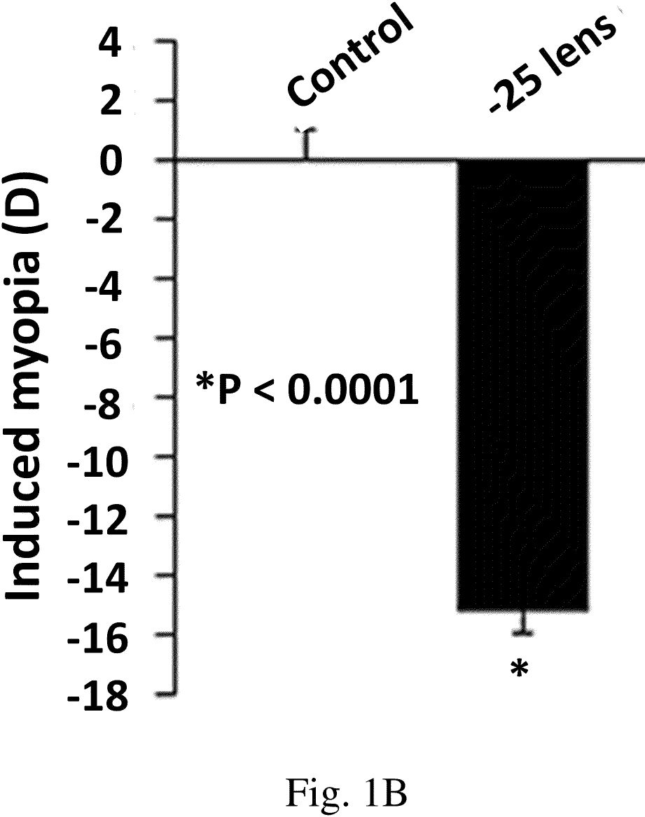

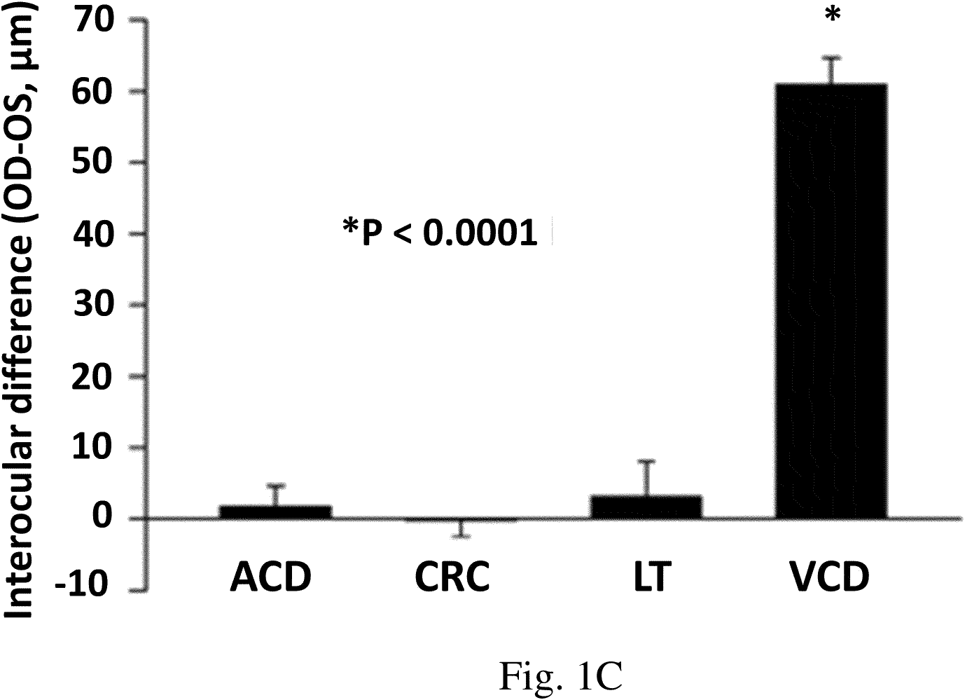

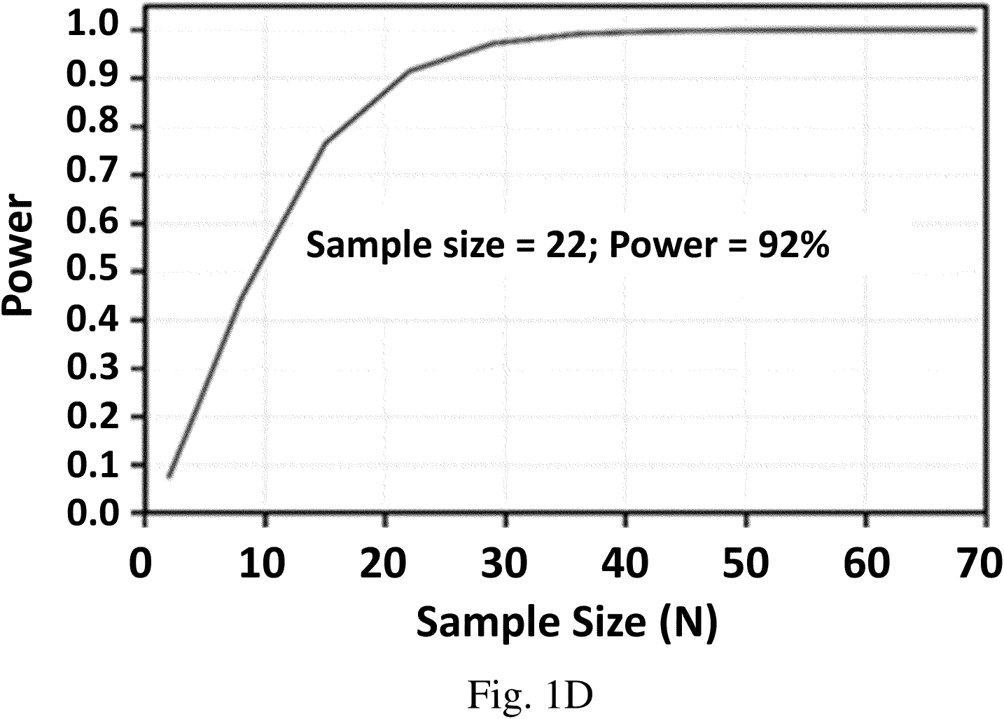

Myopia was induced in 24-days old C57BL/6J (B6) mice by attaching -25 diopter (D) lens placed in a plastic 3D-printed frame over right eye. The contralateral eye served as control. Mice were raised with lenses for 3 weeks (FIG. 1A). After 3 weeks, the lenses were removed and refractive errors in the lens-treated eyes and contralateral control eyes were compared. Lens-treatment produced myopia in the lens-treated eyes (average refractive error = -14.6 ± 0.3 D) relative to the control eyes (average refractive error = +0.6 ± 0.6 D) (FIG. 1B); the interocular difference in refractive error (-15.2 ± 0.7 D) was highly significant (P < 0.0001). High-resolution MRI revealed enlargement of the eye and the vitreous chamber in the treated eyes. The diameter of lens-treated eyes was on average 65 ± 8 µm larger (P < 0.0001; FIG. 1C), and the vitreous chamber depth in the lens-treated eyes was 61 ± 4 µm longer (P < 0.0001; FIG. 1C), than that of the control fellow eyes. No significant interocular differences were observed in the anterior chamber depth, corneal radius of curvature and crystalline lens thickness (FIG. 1C), suggesting that changes induced in the mouse eyes treated with negative lenses, are primarily confined to the posterior segment of the eye, similar to human myopia. Statistical power analysis revealed that differences as small as 0.5 diopters in refractive error between the eyes can be identified with 90% statistical power with the sample size of 22 mice (FIG. 1D).

### Example 2. Levocabastine Suppresses Myopia in Subjects With Lens-Induced Myopia

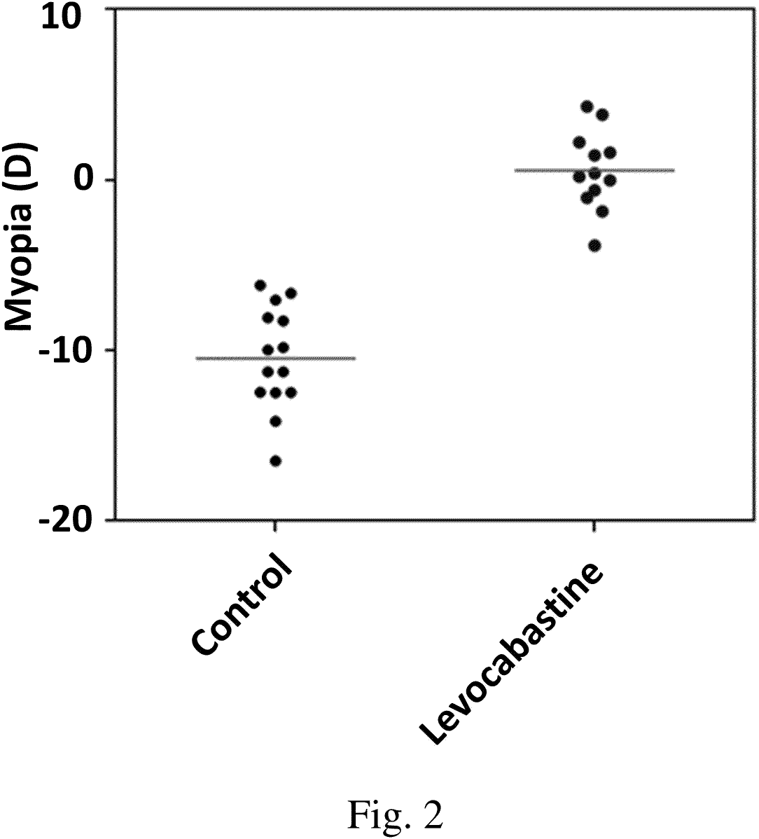

Levocabastine hydrochloride was identified as one of the top 10 drug candidates using the pharmacogenomic pipeline for anti-myopic drug development. It was discovered that systemic oral administration of levocabastine hydrochloride inhibited myopia by >100% (FIG. 2).

The experimental group of B6 mice was raised with -25 D lenses over right eye (as described in Example 1) on a diet supplemented with 5 mg/kg of levocabastine hydrochloride for 3 weeks, while the control group of B6 mice with -25 D lenses over right eye was raised on a regular non-medicated diet. The interocular difference in refractive error between lens-treated eyes and control eyes in the levocabastine-treated animals after 3 weeks of lens treatment was +0.55 ± 2.32 D versus -10.47 ± 3.02 D in the control group, P-value = 2.83 × 10⁻¹⁰. See FIG. 2.

### Example 3. Fenoterol Hydrobromide Suppresses Myopia in Subjects With Lens-Induced Myopia

Fenoterol hydrobromide was identified as one of the top 10 drug candidates using the pharmacogenomic pipeline for anti-myopic drug development. It was discovered that systemic oral administration of fenoterol hydrobromide inhibited myopia by 92% (FIG. 3).

The experimental group of B6 mice was raised with -25 D lenses over right eye on a diet supplemented with 1 mg/kg of fenoterol hydrobromide for 3 weeks, while the control group of B6 mice with -25 D lenses over right eye was raised on a regular non-medicated diet. The interocular difference in refractive error between lens-treated eyes and control eyes in the fenoterol-treated animals after 3 weeks of lens treatment was -0.8 ± 1.80 D versus -10.47 ± 3.02 D in the control group, P = 2.47 × 10⁻⁰⁸. See FIG. 3.

### Example 4. Trichostatin A Suppresses Myopia in Subjects With Lens-Induced Myopia

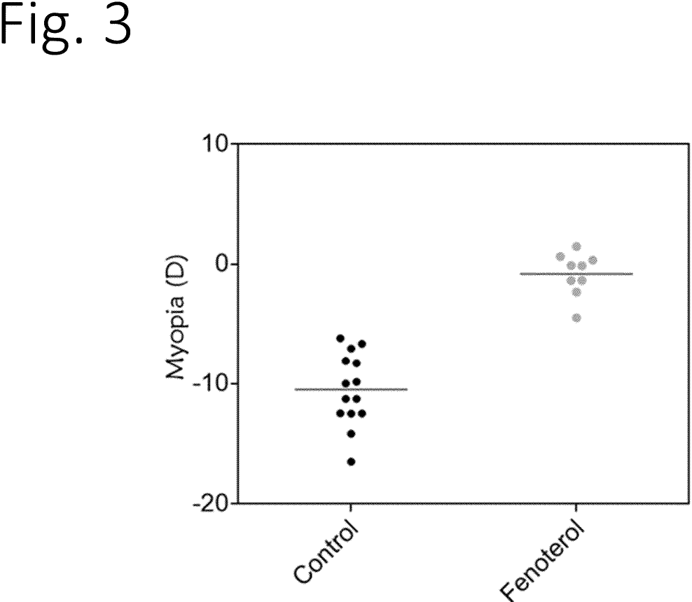

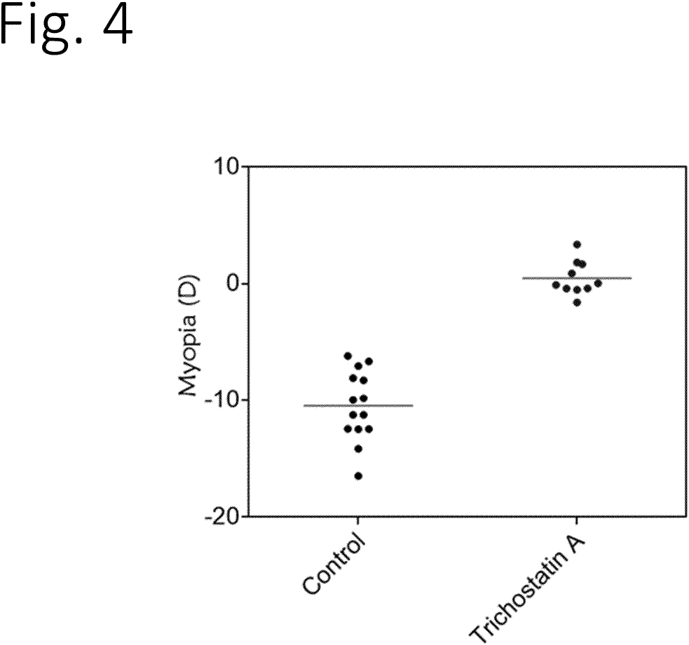

Trichostatin A was identified as one of the top 10 drug candidates using the pharmacogenomic pipeline for anti-myopia drug development. It was discovered that systemic oral administration of trichostatin A inhibited myopia by more than 100% (FIG. 4).

The experimental group of B6 mice was raised with -25 D lenses over right eye on a diet supplemented with 1 mg/kg of trichostatin A for 3 weeks, while the control group of B6 mice with -25 D lenses over right eye was raised on a regular non-medicated diet. The interocular difference in refractive error between lens-treated eyes and control eyes in the trichostatin-A-treated animals after 3 weeks of lens treatment was 0.48 ± 1.46 D versus -10.47 ± 3.02 D in the control group, P = 4.43 × 10⁻¹⁰. See FIG. 4.

### Example 5. Fingolimod Phosphate Suppresses Myopia in Subjects With Lens-Induced Myopia

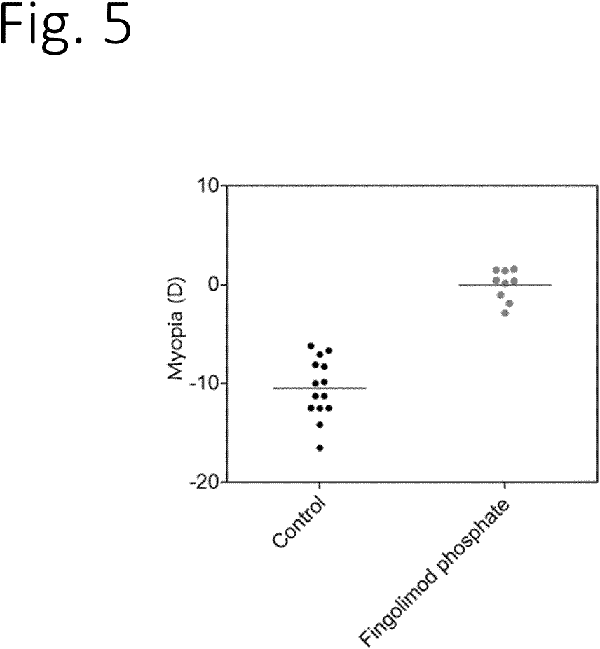

Fingolimod phosphate was identified as one of the top 10 drug candidates using the pharmacogenomic pipeline for anti-myopia drug development. It was discovered that systemic oral administration of fingolimod phosphate inhibited myopia by approximately 100% (FIG. 5).

The experimental group of B6 mice was raised with -25 D lenses over right eye on a diet supplemented with 0.3 mg/kg of fingolimod phosphate for 3 weeks, while the control group of B6 mice with -25 D lenses over right eye was raised on a regular non-medicated diet. The interocular difference in refractive error between lens-treated eyes and control eyes in the fingolimod-treated animals after 3 weeks of lens treatment was -0.02 ± 1.58 D versus -10.47 ± 3.02 D in the control group, P = 4.63 × 10⁻⁹. See FIG. 5.

### Example 6. Berberine Chloride Suppresses Myopia in Subjects With Lens-Induced Myopia

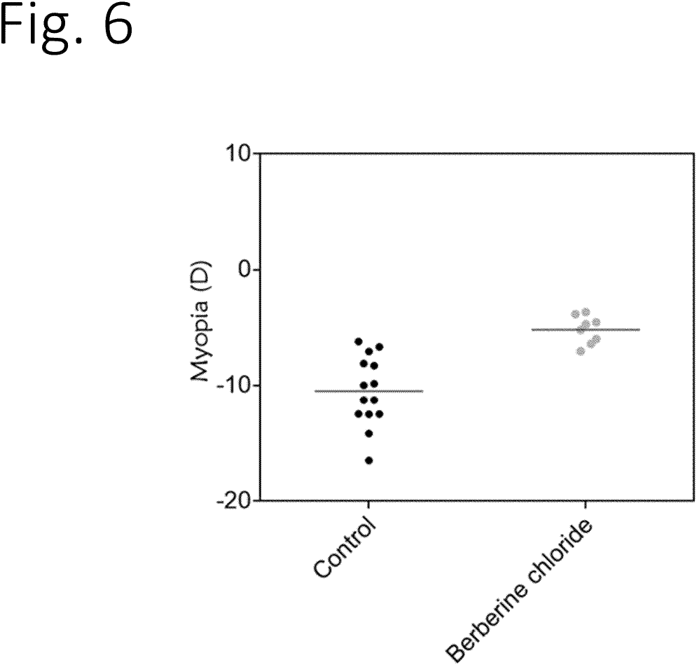

Berberine chloride was identified as one of the top 10 drug candidates using the pharmacogenomic pipeline for anti-myopia drug development. It was discovered that systemic oral administration of berberine chloride inhibited myopia by approximately 50% (FIG. 6).

The experimental group of B6 mice was raised with -25 D lenses over right eye on a diet supplemented with 20 mg/kg of berberine chloride for 3 weeks, while the control group of B6 mice with -25 D lenses over right eye was raised on a regular non-medicated diet. The interocular difference in refractive error between lens-treated eyes and control eyes in the berberine-treated animals after 3 weeks of lens treatment was -5.15 ± 1.22 D versus -10.47 ± 3.02 D in the control group, P = 1.31 × 10⁻⁴. See FIG. 6.

### Example 7. S-Methyl-L-Thiocitrulline Hydrochloride Suppresses Myopia in Subjects With Lens-Induced Myopia

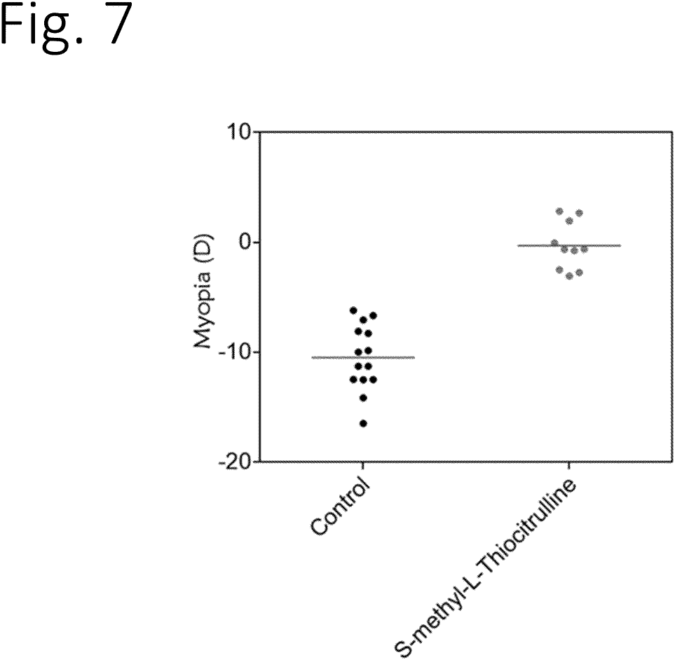

S-methyl-L-thiocitrulline hydrochloride was identified as one of the top 10 drug candidates using the pharmacogenomic pipeline for anti-myopia drug development. It was discovered that systemic oral administration of S-methyl-L-thiocitrulline hydrochloride inhibited myopia by approximately 97% (FIG. 7).

The experimental group of B6 mice was raised with -25 D lenses over right eye on a diet supplemented with 10 mg/kg of S-methyl-L-thiocitrulline hydrochloride for 3 weeks, while the control group of B6 mice with -25 D lenses over right eye was raised on a regular non-medicated diet. The interocular difference in refractive error between lens-treated eyes and control eyes in the S-methyl-L-thiocitrulline-treated animals after 3 weeks of lens treatment was -0.28 ± 2.17 D versus -10.47 ± 3.02 D in the control group, P = 6.73 × 10⁻⁹. See FIG. 7.

### REFERENCES

1. Kempen JH, Mitchell P, Lee KE, et al. The prevalence of refractive errors among adults in the United States, Western Europe, and Australia. Arch Ophthalmol 2004;122:495-505.
2. Vitale S, Sperduto RD, Ferris FL, 3rd. Increased prevalence of myopia in the United States between 1971-1972 and 1999-2004. Arch Ophthalmol 2009;127:1632-1639.
3. Lin LL, Shih YF, Hsiao CK, Chen CJ. Prevalence of myopia in Taiwanese schoolchildren: 1983 to 2000. Ann Acad Med Singapore 2004;33:27-33.
4. Holden BA, Fricke TR, Wilson DA, et al. Global Prevalence of Myopia and High Myopia and Temporal Trends from 2000 through 2050. Ophthalmology 2016;123:1036-1042.
5. Saw SM, Gazzard G, Shih-Yen EC, Chua WH. Myopia and associated pathological complications. Ophthalmic Physiol Opt 2005;25:381-391.
6. Verhoeven VJ, Wong KT, Buitendijk GH, Hofman A, Vingerling JR, Klaver CC. Visual consequences of refractive errors in the general population. Ophthalmology 2015;122:101-109.
7. Qiu M, Wang SY, Singh K, Lin SC. Association between myopia and glaucoma in the United States population. Invest Ophthalmol Vis Sci 2013;54:830-835.
8. Praveen MR, Vasavada AR, Jani UD, Trivedi RH, Choudhary PK. Prevalence of cataract type in relation to axial length in subjects with high myopia and emmetropia in an Indian population. Am J Ophthalmol 2008;145:176-181.
9. Pizzarello L, Abiose A, Ffytche T, et al. VISION 2020: The Right to Sight: a global initiative to eliminate avoidable blindness. Arch Ophthalmol 2004;122:615-620.
10. Wojciechowski R. Nature and nurture: the complex genetics of myopia and refractive error. Clin Genet 2011;79:301-320.
11. Parssinen O, Lyyra AL. Myopia and myopic progression among schoolchildren: a three-year follow-up study. Invest Ophthalmol Vis Sci 1993;34:2794-2802.
12. Goss DA. Nearwork and myopia. Lancet 2000;356:1456-1457.
13. Saw SM, Chua WH, Hong CY, et al. Nearwork in early-onset myopia. Invest Ophthalmol Vis Sci 2002;43:332-339.
14. Gwiazda J, Thorn F, Bauer J, Held R. Myopic children show insufficient accommodative response to blur. Invest Ophthalmol Vis Sci 1993;34:690-694.
15. Seidemann A, Schaeffel F. An evaluation of the lag of accommodation using photorefraction. Vision Res 2003;43:419-430.
16. Charman WN. Near vision, lags of accommodation and myopia. Ophthalmic Physiol Opt 1999;19:126-133.
17. Gwiazda JE, Hyman L, Norton TT, et al. Accommodation and related risk factors associated with myopia progression and their interaction with treatment in COMET children. Invest Ophthalmol Vis Sci 2004;45:2143-2151.
18. Zylbermann R, Landau D, Berson D. The influence of study habits on myopia in Jewish teenagers. Journal of pediatric ophthalmology and strabismus 1993;30:319-322.
19. Williams C, Miller LL, Gazzard G, Saw SM. A comparison of measures of reading and intelligence as risk factors for the development of myopia in a UK cohort of children. Br J Ophthalmol 2008;92:1117-1121.
20. Pan CW, Ramamurthy D, Saw SM. Worldwide prevalence and risk factors for myopia. Ophthalmic Physiol Opt 2012;32:3-16.
21. Saw SM, Cheng A, Fong A, Gazzard G, Tan DT, Morgan I. School grades and myopia. Ophthalmic Physiol Opt 2007;27:126-129.
22. Zadnik K, Mutti DO. Refractive error changes in law students. Am J Optom Physiol Opt 1987;64:558-561.
23. Ip JM, Rose KA, Morgan IG, Burlutsky G, Mitchell P. Myopia and the urban environment: findings in a sample of 12-year-old Australian school children. Invest Ophthalmol Vis Sci 2008;49:3858-3863.
24. Ting PW, Lam CS, Edwards MH, Schmid KL. Prevalence of myopia in a group of Hong Kong microscopists. Optom Vis Sci 2004;81:88-93.
25. Troilo D, Smith EL, 3rd, Nickla DL, et al. IMI - Report on Experimental Models of Emmetropization and Myopia. Invest Ophthalmol Vis Sci 2019;60:M31-M88.
26. Huang HM, Chang DS, Wu PC. The Association between Near Work Activities and Myopia in Children-A Systematic Review and Meta-Analysis. PLoS One 2015;10:e0140419.
27. Tedja MS, Haarman AEG, Meester-Smoor MA, et al. IMI - Myopia Genetics Report. Invest Ophthalmol Vis Sci 2019;60:M89-M105.
28. Dirani M, Shekar SN, Baird PN. Evidence of shared genes in refraction and axial length: the Genes in Myopia (GEM) twin study. Invest Ophthalmol Vis Sci 2008;49:4336-4339.
29. Jones-Jordan LA, Sinnott LT, Manny RE, et al. Early childhood refractive error and parental history of myopia as predictors of myopia. Invest Ophthalmol Vis Sci 2010;51:115-121.
30. Dirani M, Shekar SN, Baird PN. Adult-onset myopia: the Genes in Myopia (GEM) twin study. Invest Ophthalmol Vis Sci 2008;49:3324-3327.
31. Tsai MY, Lin LL, Lee V, Chen CJ, Shih YF. Estimation of heritability in myopic twin studies. Japanese journal of ophthalmology 2009;53:615-622.
32. Lyhne N, Sjolie AK, Kyvik KO, Green A. The importance of genes and environment for ocular refraction and its determiners: a population based study among 20-45 year old twins. Br J Ophthalmol 2001;85:1470-1476.
33. Tkatchenko TV, Shah RL, Nagasaki T, Tkatchenko AV. Analysis of genetic networks regulating refractive eye development in collaborative cross progenitor strain mice reveals new genes and pathways underlying human myopia. BMC Med Genomics 2019;12:113.
34. Tkatchenko AV, Tkatchenko TV, Guggenheim JA, et al. APLP2 Regulates Refractive Error and Myopia Development in Mice and Humans. PLoS Genet 2015;11:e1005432.
35. Shelton L, Troilo D, Lerner MR, Gusev Y, Brackett DJ, Rada JS. Microarray analysis of choroid/RPE gene expression in marmoset eyes undergoing changes in ocular growth and refraction. Mol Vis 2008;14:1465-1479.
36. Tkatchenko AV, Walsh PA, Tkatchenko TV, Gustincich S, Raviola E. Form deprivation modulates retinal neurogenesis in primate experimental myopia. Proc Natl Acad Sci U S A 2006;103:4681-4686.
37. Tkatchenko TV, Troilo D, Benavente-Perez A, Tkatchenko AV. Gene expression in response to optical defocus of opposite signs reveals bidirectional mechanism of visually guided eye growth. PLoS biology 2018;16:e2006021.
38. Smith EL, 3rd. Prentice Award Lecture 2010: A case for peripheral optical treatment strategies for myopia. Optom Vis Sci 2011;88:1029-1044.
39. Troilo D, Gottlieb MD, Wallman J. Visual deprivation causes myopia in chicks with optic nerve section. Curr Eye Res 1987;6:993-999.
40. Fan DS, Lam DS, Lam RF, et al. Prevalence, incidence, and progression of myopia of school children in Hong Kong. Invest Ophthalmol Vis Sci 2004;45:1071-1075.
41. Donovan L, Sankaridurg P, Ho A, Naduvilath T, Smith EL, 3rd, Holden BA. Myopia progression rates in urban children wearing single-vision spectacles. Optom Vis Sci 2012;89:27-32.
42. Cortinez MF, Chiappe JP, Iribarren R. Prevalence of refractive errors in a population of office-workers in Buenos Aires, Argentina. Ophthalmic epidemiology 2008;15:10-16.
43. Fernandez-Montero A, Olmo-Jimenez JM, Olmo N, et al. The impact of computer use in myopia progression: a cohort study in Spain. Prev Med 2015;71:67-71.
44. Ong E, Grice K, Held R, Thorn F, Gwiazda J. Effects of spectacle intervention on the progression of myopia in children. Optom Vis Sci 1999;76:363-369.
45. Walline JJ, Jones LA, Sinnott L, et al. A randomized trial of the effect of soft contact lenses on myopia progression in children. Invest Ophthalmol Vis Sci 2008;49:4702-4706.
46. Wildsoet CF, Chia A, Cho P, et al. IMI - International Myopia Institute: Interventions for Controlling Myopia Onset and Progression Report. Invest Ophthalmol Vis Sci 2019;60:M106-M131.
47. Gwiazda JE, Hyman L, Everett D, Norton T, Kurtz D, Manny R. Five-year results from the correction of myopia evaluation trial (COMET). Investigative Ophthalmology and Visual Science 2006;47: E-abstract 1166.
48. Sun Y, Xu F, Zhang T, et al. Orthokeratology to control myopia progression: a meta-analysis. PLoS One 2015;10:e0124535.
49. Si JK, Tang K, Bi HS, Guo DD, Guo JG, Wang XR. Orthokeratology for myopia control: a meta-analysis. Optom Vis Sci 2015;92:252-257.
50. Cho P, Cheung SW. Discontinuation of orthokeratology on eyeball elongation (DOEE). Cont Lens Anterior Eye 2017;40:82-87.
51. Woods J, Guthrie SE, Keir N, et al. Inhibition of defocus-induced myopia in chickens. Invest Ophthalmol Vis Sci 2013;54:2662-2668.
52. Walline JJ, Greiner KL, McVey ME, Jones-Jordan LA. Multifocal contact lens myopia control. Optom Vis Sci 2013;90:1207-1214.
53. Paune J, Morales H, Armengol J, Quevedo L, Faria-Ribeiro M, Gonzalez-Meijome JM. Myopia Control with a Novel Peripheral Gradient Soft Lens and Orthokeratology: A 2-Year Clinical Trial. BioMed research international 2015;2015:507572.
54. Li SM, Kang MT, Wu SS, et al. Studies using concentric ring bifocal and peripheral add multifocal contact lenses to slow myopia progression in school-aged children: a meta-analysis. Ophthalmic Physiol Opt 2017;37:51-59.
55. Chua WH, Balakrishnan V, Chan YH, et al. Atropine for the treatment of childhood myopia. Ophthalmology 2006;113:2285-2291.
56. Tong L, Huang XL, Koh AL, Zhang X, Tan DT, Chua WH. Atropine for the treatment of childhood myopia: effect on myopia progression after cessation of atropine. Ophthalmology 2009;116:572-579.
57. Chia A, Chua WH, Cheung YB, et al. Atropine for the treatment of childhood myopia: safety and efficacy of 0.5%, 0.1%, and 0.01% doses (Atropine for the Treatment of Myopia 2). Ophthalmology 2012;119:347-354.
58. Chia A, Chua WH, Wen L, Fong A, Goon YY, Tan D. Atropine for the treatment of childhood myopia: changes after stopping atropine 0.01%, 0.1% and 0.5%. Am J Ophthalmol 2014;157:451-457 e451.
59. Chia A, Lu QS, Tan D. Five-Year Clinical Trial on Atropine for the Treatment of Myopia 2: Myopia Control with Atropine 0.01% Eyedrops. Ophthalmology 2016;123:391-399.
60. Yam JC, Jiang Y, Tang SM, et al. Low-Concentration Atropine for Myopia Progression (LAMP) Study: A Randomized, Double-Blinded, Placebo-Controlled Trial of 0.05%, 0.025%, and 0.01% Atropine Eye Drops in Myopia Control. Ophthalmology 2019;126:113-124.
61. Whatham AR, Lunn D, Judge SJ. Effects of Monocular Atropinization on Refractive Error and Eye Growth in Infant New World Monkeys. Invest Ophthalmol Vis Sci 2019;60:2623-2630.
62. Trier K, Olsen EB, Kobayashi T, Ribel-Madsen SM. Biochemical and ultrastructural changes in rabbit sclera after treatment with 7-methylxanthine, theobromine, acetazolamide, or L-ornithine. Br J Ophthalmol 1999;83:1370-1375.
63. Trier K, Munk Ribel-Madsen S, Cui D, Brogger Christensen S. Systemic 7-methylxanthine in retarding axial eye growth and myopia progression: a 36-month pilot study. J Ocul Biol Dis Infor 2008;1:85-93.
64. Cui D, Trier K, Zeng J, et al. Effects of 7-methylxanthine on the sclera in form deprivation myopia in guinea pigs. Acta Ophthalmol 2011;89:328-334.
65. Nie HH, Huo LJ, Yang X, et al. Effects of 7-methylxanthine on form-deprivation myopia in pigmented rabbits. International journal of ophthalmology 2012;5:133-137.
66. Hung LF, Arumugam B, Ostrin L, et al. The Adenosine Receptor Antagonist, 7-Methylxanthine, Alters Emmetropizing Responses in Infant Macaques. Invest Ophthalmol Vis Sci 2018;59:472-486.
67. Cottriall CL, McBrien NA. The M1 muscarinic antagonist pirenzepine reduces myopia and eye enlargement in the tree shrew. Invest Ophthalmol Vis Sci 1996;37:1368-1379.
68. Cottriall CL, Truong HT, McBrien NA. Inhibition of myopia development in chicks using himbacine: a role for M(4) receptors? Neuroreport 2001;12:2453-2456.
69. Siatkowski RM, Cotter SA, Crockett RS, et al. Two-year multicenter, randomized, double-masked, placebo-controlled, parallel safety and efficacy study of 2% pirenzepine ophthalmic gel in children with myopia. Journal of AAPOS : the official publication of the American Association for Pediatric Ophthalmology and Strabismus / American Association for Pediatric Ophthalmology and Strabismus 2008;12:332-339.
70. Stone RA, Liu J, Sugimoto R, Capehart C, Zhu X, Pendrak K. GABA, experimental myopia, and ocular growth in chick. Invest Ophthalmol Vis Sci 2003;44:3933-3946.
71. Cheng ZY, Wang XP, Schmid KL, Han XG. Inhibition of form-deprivation myopia by a GABAAOr receptor antagonist, (1,2,5,6-tetrahydropyridin-4-yl) methylphosphinic acid (TPMPA), in guinea pigs. Graefes Arch Clin Exp Ophthalmol 2014;252:1939-1946.
72. Cheng ZY, Wang XP, Schmid KL, et al. GABAB receptor antagonist CGP46381 inhibits form-deprivation myopia development in guinea pigs. BioMed research international 2015;2015:207312.
73. Carr BJ, Nguyen CT, Stell WK. Alpha2 -adrenoceptor agonists inhibit form-deprivation myopia in the chick. Clin Exp Optom 2019;102:418-425.
74. Liu Y, Wang Y, Lv H, Jiang X, Zhang M, Li X. alpha-adrenergic agonist brimonidine control of experimentally induced myopia in guinea pigs: A pilot study. Mol Vis 2017;23:785-798.
75. Iuvone PM, Tigges M, Stone RA, Lambert S, Laties AM. Effects of apomorphine, a dopamine receptor agonist, on ocular refraction and axial elongation in a primate model of myopia. Invest Ophthalmol Vis Sci 1991;32:1674-1677.
76. Yan T, Xiong W, Huang F, et al. Daily Injection But Not Continuous Infusion of Apomorphine Inhibits Form-Deprivation Myopia in Mice. Invest Ophthalmol Vis Sci 2015;56:2475-2485.
77. El-Nimri NW, Wildsoet CF. Effects of Topical Latanoprost on Intraocular Pressure and Myopia Progression in Young Guinea Pigs. Invest Ophthalmol Vis Sci 2018;59:2644-2651.
78. Mori K, Kurihara T, Miyauchi M, et al. Oral crocetin administration suppressed refractive shift and axial elongation in a murine model of lens-induced myopia. Sci Rep 2019;9:295.
79. Wong YL, Sabanayagam C, Ding Y, et al. Prevalence, Risk Factors, and Impact of Myopic Macular Degeneration on Visual Impairment and Functioning Among Adults in Singapore. Invest Ophthalmol Vis Sci 2018;59:4603-4613.
80. Tkatchenko TV, Tkatchenko AV. Pharmacogenomic approach to antimyopia drug development: pathways lead the way. Trends Pharmacol Sci 2019;40:834-853.

1. A method of preventing or treating myopia in a subject in need thereof comprising administering to the subject a therapeutically effective amount of a composition comprising an active drug compound selected from the group consisting of levocabastine hydrochloride, fenoterol hydrobromide, trichostatin A, fingolimod (FTY720) phosphate, berberine chloride, S-methyl-L-thiocitrulline hydrochloride, and derivatives thereof.

2. The method of claim 1, wherein the subject is about 4 years of age to about 30 years of age.

3. The method of claim 1, wherein the subject is about 6 years of age to about 20 years of age.

4. The method of claim 1, comprising administering the composition to the subject once a day.

5. The method of claim 1, comprising administering the composition to the subject about once, twice, or three times a week.

6. The method of claim 1, comprising administering the composition to the subject continuously or intermittently for about 5 years to about 10 years.

7. The method of claim 1, wherein the subject is monitored for suppression of myopia and the therapeutically effective amount or frequency of administration is adjusted depending on the degree of suppression.

8. The method of claim 1, wherein the composition is administered orally, via eye drops, via injection, via patch or through a contact lens.

9. The method of claim 1, wherein the composition is in an extended release form.

10. The method of claim 8, wherein the contact lens is chosen from the group consisting of a plano contact lens, a single-vision contact lens and a multi-focal contact lens.

11. The method of claim 8, wherein the composition is loaded on the internal surface of the lens or the entire volume of the contact lens.

12. The method of claim 1, wherein the composition is in an extended drug release formulation or composition.

13. The method of claim 12, wherein the extended drug release formulation or composition is selected from the group consisting of nanosponge, patch, and gel.

14. The method of claim 1, wherein the composition further comprises excipients or additional agents which suppress, prevent or treat myopia.

15. The method of claim 1, wherein the active drug compound is modified to improve its efficacy, penetration through ocular tissues, stability and/or bioavailability, and/or reduce side effects.
